(19) United States
(12) Patent Application Publication
(10) Pub. No.: US 2011/0236923 A1
(43) Pub. Date: Sep. 29, 2011

(54) METHOD FOR STAINING AND SORTING OF A SMALL VOLUME OF SPERM

(75) Inventor: Sayed Bahawaddin HASHEMI, Herndon, VA (US)

(73) Assignee: GENETICS & IVF INSTITUTE

(21) Appl. No.: 12/729,863

(22) Filed: Mar. 23, 2010

(51) Int. Cl. C12Q 1/04

(52) U.S. Cl. 435/34

(57) ABSTRACT

The present invention relates to a method of sorting of a small volume or low concentration stained sperm sample, the method includes: (a) diluting the stained sperm cells in a ratio of 1:1 or less by volume with a dilution buffer to obtain a diluted volume of said stained sperm sample; (b) placing the diluted volume of the stained sperm sample on a flow cytometer to initiate the sorting process; (c) sorting the stained sperm cells in the diluted volume based upon a preselected sperm cell or stain characteristic; and (d) collecting sorted sperm cells.

### FIELD OF THE INVENTION

The present invention relates to the field of sperm preparation, staining, and sorting.

### BACKGROUND OF THE INVENTION

In flow cytometric sperm sorting, sperm are conveyed, under pressure, from the sample container (usually a round-bottom centrifuge tube) to the flow cytometer via small diameter tubing. One end of the sample tubing is connected to the flow cytometer and the other end is situated in the sample container with its opening just above the bottom of the sample container. A significant problem with conventional sperm sorting procedures is that not all of the sperm in a sample are recoverable for sorting, which reduces the sperm number and/or volume and can lead to low volume or low concentration sperm samples that cannot be sorted. Often insufficient viable sperm cells are present in a sample or the optimal sperm concentration for sperm staining is in a volume inadequate for sorting. These problems can be due to, for example, the freeze-thawing of a stored semen sample or a fresh semen sample with low sperm count.

Thus, there exists a need for an improved sperm sorting method for sorting of a low volume or low concentration sperm sample and obtaining the desired number of sorted sperm.

### SUMMARY OF THE INVENTION

In one aspect, the present invention relates generally to a method of sorting of a small volume or low concentration stained sperm sample that has been exposed to predetermined staining conditions of stain concentration, sperm numbers, staining time and staining temperature. The method includes the steps of: (a) diluting the stained sperm cells in a ratio of 1:1 or less by volume with a dilution buffer to obtain a diluted volume of said stained sperm sample; (b) placing the diluted volume of the stained sperm sample onto a flow cytometer to initiate the sorting process; (c) sorting the stained sperm cells in the diluted volume based upon a preselected sperm cell or stain characteristic; and (d) collecting sorted sperm cells.

In one aspect, the present invention relates generally to a method of sorting sperm cells, including (a) obtaining a sperm sample which contains a plurality of sperm cells in a volume of 0.3 ml or less or wherein the plurality of sperm cells is less than about 2×10⁶ cells/ml; (b) staining the sperm cells by forming a staining mixture comprising intact viable sperm cells and a DNA selective fluorescent dye for a period of time sufficient to allow the dye to bind to DNA of the sperm cells; (c) diluting the stained sperm cells of step (b) to obtain a diluted volume of said stained sperm cells; (d) sorting the stained sperm cells in the diluted volume based upon a preselected sperm cell characteristic; and (e) collecting sorted sperm cells.

In one embodiment, the sperm sample is a freshly obtained mammalian semen or a cryopreserved and subsequently thawed mammalian semen specimen. In one embodiment, the sperm cells are from a mammal including a human, bovine, equine, ovine, caprine, porcine, canine, feline, cervine, lagomorph, cavid or marine species of mammals.

In one embodiment, the stained sperm sample contains less than about 2×10⁶ sperm cells/mL; or the stained sperm sample contains less than about 1×10⁶ sperm cells/ml, or the stained sperm sample contains less than about 5×10⁵ sperm cells/ml. In one embodiment, the volume of the stained sperm sample is less than about 0.3 mL; or the volume of the stained sperm sample is less than about 0.25 mL, or the volume of the stained sperm sample is less than about 0.2 mL.

In one embodiment, the diluting step includes diluting the stained sperm cells with a buffer. In another embodiment, the buffer is modified Ham's F-10 (MHF-10) or sucrose wash medium (SWM).

### DETAILED DESCRIPTION OF THE INVENTION

Currently, the most effective sperm sorting method is flow cytometry, the measurement and sorting of X and Y sperm cells in a moving liquid stream. In the apparatus used in cytometry, called a cytometer or flow cytometer, a fluid flows through a sensing orifice in the presence of an electric field. Once a particle (e.g., a sperm cell) enters the orifice it will disturb the electric field, increasing the resistance of the circuit and the size of the disturbance will be proportional to the volume and relative particle size to the volume of the orifice. An electric pulse is caused as each particle passes through the orifice, and the pulse height will be proportional to the size of the particle. Using sophisticated focusing techniques to enhance the resolution, the flow cytometer can be used for electrical sizing of the cells or differentiating dead cells from the live cells. If the cells are chemically tagged with fluorescent molecules when they are flowed past a source of monochromatic light (a laser), a lens system can be used to detect the amount of light scattered by the cells as well as the fluorescent signals emitted by the fluorescently-tagged cells. The scattered light intensity is a complex function of cell size and refractive properties of the cell but generally it can be used as a way of measuring the cell area. The wide angle scatter of the light can be used as a tool for distinguishing cells of equal size which have different internal refraction properties. For example, dead sperm cells, due to having lost cytoplasm due to cell membrane disruption, scatter less light at right angles than viable sperm cells. The fluorescent signals are roughly a measure of the concentration of the fluorescent molecule in a cell or on its surface. Sperm cells, for example, contain half of each chromosome pair and one of the sex chromosomes: 22 autosomes and an X or a Y chromosome. Since the X chromosome is larger than the Y chromosome, the X chromosome contains more DNA than a Y chromosome. Thus, X-bearing sperm cells contain more chromosomal DNA than Y-bearing sperm cells and, therefore, can take up more DNA-specific fluorescent dye and emit more fluorescence than Y-bearing sperm cells. Therefore, the flow cytometer is an instrument for rapid and accurate measurement of particle volume (electrical resistance sizing), particle area (forward light scatter), fluorescence intensity (fluorescent emission), and particle granularity (right angle light scatter). For sperm sorting purposes, measuring particle area, size and granularity allows for differentiation of dead and alive sperm cells while measuring fluorescent intensity helps to differentiate characteristics among cells, in one case, DNA content, and sort the X- and Y-bearing sperm cells exhibiting the desired fluorescence characteristics. Normally, a charging pulse is applied to the sperm flow stream, by which X and Y sperm cells are selectively deflected into distinct streams and the sperm cells in those streams are counted.

A significant problem with conventional sperm sorting procedures is that it is not possible to recover all of the sperm in a semen sample for sorting. This is because sorting of a particular aliquot is halted before the level of a sperm suspension falls below the opening of the sample tubing within the sample container. Otherwise, if sorting were to continue, the resultant aspiration of the pressurizing gas into the sample tubing would introduce bubbles in the fluidics stream, disrupting the fluidics stability of the sorting instrument. Thus, there is always a volume of sperm suspension remaining in the sample container. This residual sperm suspension contains functional sperm that could otherwise be sorted. This creates a problem when sorting sperm samples in which there are a very low number of sperm cells available to be sorted. It also creates a problem when sorting sperm samples in which sperm numbers are adequate, but the percentage of viable sperm is low. The result is a low volume or low concentration sperm sample, which can run out during the sorting process before the desired number of sperm cells is collected.

Contrary to conventional wisdom that sperm cell viability decreases with an increase in dilution (See, for example, Garner et al. Journal of Andrology, Vol. 19, No. 3 (1997)), applicant has discovered that further diluting of a stained sperm suspension surprisingly increases the yield of the viable sperm cells sorted or collected irrespective of the volume or sperm cell concentration of the initial stained sample. It has further been discovered that the differential fluorescence emitted by viable X- and Y-bearing sperm cells can be maintained or improved by diluting the stained sperm suspension with specific buffers and up to certain dilution ratios prior to or during sorting. It has unexpectedly been observed that no leaching or bleaching of the fluorescent dye occurs and the fluorescent signal emitted by the sperm cells is adequate for sorting if the specific dilution parameters taught by the present invention are maintained.

According to one aspect of the present invention, a stained sperm sample with small volume of, for example, less than about 0.3 mL or a sperm sample with sperm concentration of, for example, less than about 2×10⁶ sperm cells/mL is diluted further before or during the sorting process. In one embodiment, the dilution of the sperm sample is carried out after staining of the sperm and before the sorting process. In another embodiment, the dilution of the stained sperm suspension is carried out during the sorting process. In one embodiment, the stained sperm suspension is diluted to a volume less than or equal to 1:1 ratio by volume, of the dilution buffer to stained sperm suspension, respectively. In one embodiment, the dilution buffer used is modified Ham's F-10 (MHF-10) or sperm washing medium (SWM) or other medium routinely used in the preparation/dilution of sperm.

The methods of the present invention can be applied to sperm cells from human, bovine, equine, ovine, swine, canine, feline, lagomorph, cavid, or other mammals including the marine mammals. The methods disclosed herein may be applied either to sperm recently obtained from the particular source (i.e., obtained from human, bovine, porcine, or other mammals within minutes or hours before staining) or to sperm cells that have been cryopreserved and subsequently thawed. In one embodiment, the sperm sample is a fresh mammalian semen sample or a cryopreserved and subsequently thawed human semen sample.

The sperm cells can be derived from a freshly obtained semen sample or from a thawed cryopreserved semen sample. Various methods of collection of viable sperm are known and include, for example, an artificial vagina method, electroejaculation, or a gloved hand method. Cryopreserved semen samples can be thawed by various techniques such as with ram spermatozoa or as a glass tube shaken in a 37° C. water bath.

The methods of the present invention facilitate sorting of the low volume or low concentration samples of stained sperm cells for use in intrauterine insemination (IUI), in-vitro fertilization (IVF)/intra-cytoplasmic sperm injection (ICSI) or for cryopreservation and storage for subsequent use in IUI or IVF/ICSI, for example. Staining of sperm cells can be carried out in variety of ways known in the art. Exemplary staining methods are provided in U.S. Pat. No. 7,335,507, the entire content of which is incorporated herein by reference. For example, a sperm sample can be combined with a DNA selective dye, such as Hoechst 33342 or Hoechst 33258, each of which is commercially available from Sigma-Aldrich (St. Louis, Mo.). In general, sperm cells are stained by forming a staining mixture which comprises sperm cells and a DNA selective dye. Sperm cells are somewhat sensitive to significant changes in osmotic pressure and pH. To minimize impact upon sperm viability, therefore, it is generally preferred that the staining mixture be formed with these considerations in mind. Consistent with these considerations, the staining mixture may be formed in a variety of manners.

For example, a sperm sample is combined with a buffer to form a sperm suspension and the sperm suspension is thereafter combined with the dye to form the staining mixture. Suspending the sperm cells in a buffered liquid prior to contact with the dye can advantageously protect the sperm cells from significant changes in pH and osmotic pressure which would otherwise result from the addition of the dye. The sperm source may be fresh or thawed sperm cells. In another example, the DNA selective dye is combined with a buffer, or is included as part of a buffer recipe, thereby forming a buffered dye solution. Thus, for example, dye in the form of a neat solid, including a free-flowing powder, or a liquid composition may be combined with the buffer, or any constituents of the buffer recipe prior to the combination thereof, to form a buffered dye solution, which may then be combined with a sperm suspension.

A variety of biological buffers may be used, individually or in combination, to protect the sperm cells from significant changes in pH and osmotic pressure during staining. Typically, these buffers will have a salt concentration of about 0.001M to about 1.0M and at a pH between about 4.5 to about 8.5. Preferred buffers include Sodium Bicarbonate, TCA, TES, TRIS, TEST, sodium citrate, and HEPES.

The concentration of the dye in the staining mixture is selected such that a sufficient amount of dye will bind to the DNA to enable a mixture of X and Y chromosome-bearing sperm cells to be sorted into enriched populations of X- or Y-bearing sperm. The pH of the staining mixture is preferably maintained in the range of about 6.0 to about 8.0. Certain dyes are capable of permeating the sperm cells and specifically binding the DNA without further intervention to increase the permeability of the cells. With other dyes, however, it may be desirable to treat the sperm cells prior to staining to increase the rate of permeation without unacceptably reducing viability or motility. Any suitable method known to those skilled in the art may be used. Such methods include electroporation, the use of cell-permeation-enhancing solutions, e.g., mild surfactants, or chemical shock. Where it is desired or advantageous to use other or more stringent techniques, such treatments can include the use of liposomes or many of the techniques which are used by those skilled in the art to introduce stains, dyes, genes, or vectors into living cells. These methods include, but are not limited to microinjection such as used by Gordon et al. (Proc. Natl. Acad. Sci., 1980).

Uptake of dye by the sperm cells in the staining mixture is allowed to continue for a period of time sufficient to obtain the desired degree of DNA staining. In general, the uptake period will be between about 1 and about 160 minutes. The staining mixture may be subjected to an elevated temperature of the present invention for the entire uptake period or for a fraction thereof. The sperm-stain mixture may be agitated during the staining period. In any event, the conditions during the uptake period is sufficient for the dye to bind to the DNA such that X and Y chromosome-bearing sperm cells can be sorted based upon the differing and measurable fluorescence intensity between the X and Y chromosome-bearing sperm. Various dyes that are suitable for sperm staining are, for example, bisbenzimides such as Hoechst 33342 and Hoechst 33258, each of which is commercially available from Cal-Biochem (San Diego). Advantageously, for example, Hoechst 33342 has a low toxicity, is sufficiently cell permeable, is specific for DNA, has a fluorescence that is dramatically enhanced after binding to DNA, and displays a linear relationship between the intensity of the fluorescence and the amount of DNA present in a given cell or sample. Additional dyes are disclosed in, for example, U.S. Pat. Nos. 5,338,854, 4,774,339 and PCT publication WO 02/41906, herein incorporated by reference. Such dyes include, for example, the visible light excitable dye, SYBR-14, commercially available from Molecular Probes, Inc. (InVitrogen Corporation, Eugene, Oreg.) and the bisbenzimide-BODIPY® conjugate 6-{[3-((2Z)-2-{[1-(difluoroboryl)-3,5-dimethyl-1H-pyrrol-2-yl]methylene}-2H-pyrrol-5-yl)propanoy]amino}-N-(methyl {3-[({4-[6-(4-methylpiperazin-1-yl)-1H,3′H-2,5′-bibeenzimdazol-2′-yl]phen-oxy}acetyl)amino]propyl}amino)propyl]hexanamide (“BBC”).

In general, a stained sperm suspension of low volume or low concentration can result from number of scenarios. For example, the initial freshly collected semen sample may have contained a low number of sperm. The thawing or staining process may reduce the number of viable sperm cells in the sample. In other situations, for example, during the sorting process, the volume of the stained sperm suspension in the sample container may fall to 0.3 mL or less before a sufficient number of sperm cells have been collected. Instead of discarding such low volume or low concentration sperm samples, it has now been unexpectedly discovered that further diluting of these samples, after staining, will facilitate the continued sorting of the cells—in that an increased number of viable cells are collected after dilution of the sample than would have been collected without dilution—with minimal or no effect on cell viability or dye loss from the cells.

Thus, in one aspect, the present invention relates generally to a method of sorting a small volume or low concentration stained sperm sample, the method including the steps of: (a) diluting the stained sperm cells in a ratio of about 1:1 or less by volume with a dilution buffer to obtain a diluted volume of said stained sperm sample; (b) subjecting the diluted volume of the stained sperm sample to a flow cytometric sorting process; (c) sorting the stained sperm cells in the diluted volume based upon a preselected sperm cell or stain characteristic; and (d) collecting sorted sperm cells.

In another aspect, the present invention relates to a method of sorting sperm cells, including (a) obtaining a sperm sample which contains a plurality of sperm cells in a volume of 0.3 ml or less or wherein the plurality of sperm cells is less than about 2×10⁶ cells/ml; (b) staining the sperm cells by forming a staining mixture comprising intact viable sperm cells and a DNA selective fluorescent dye for a period of time sufficient to allow the dye to bind to DNA of the sperm cells; (c) diluting the stained sperm cells of step (b) to obtain a diluted volume of said stained sperm cells; (d) sorting the stained sperm cells in the diluted volume based upon a preselected sperm cell characteristic; and (e) collecting sorted sperm cells.

In one embodiment, a small or low volume sperm sample has a volume of 0.3 mL or less; or 0.25 mL or less, or 0.2 mL or less; or 0.15 mL or less; or 0.10 mL or less; irrespective of the number of sperm in that volume. In another embodiment, a low concentration sperm sample has a sperm concentration of 2×10⁶ sperm cells/mL or less; or 1.5×10⁶ sperm cells/mL or less; or 1.0×10⁶ sperm cells/mL or less; or 0.5×10⁶ sperm cells/mL or less; or 2.5×10⁵ sperm cells/mL or less; or 1.0×10⁵ sperm cells/mL or less; or 5×10⁴ sperm cells/mL or less; or 1×10⁴ sperm cells/mL or less.

In one embodiment, the stained suspension of the low volume or low concentration sperm sample is diluted to a volume less than or equal to about 1:1 ratio (by volume) of the dilution buffer to stained sperm suspension; or to a volume less than or equal to 0.8:1 ratio by volume, of the dilution buffer to stained sperm suspension; or to a volume less than or equal to 0.6:1 ratio by volume, of the dilution buffer to stained sperm suspension; or to a volume less than or equal to 0.4:1 ratio by volume, of the dilution buffer to stained sperm suspension.

A variety of biological buffers can be used for post-staining dilution, individually or in combination, to dilute a low volume or low concentration sperm sample without a significant change in pH and osmotic pressure during the dilution process of the present invention. Typically, these diluting buffers will have a salt concentration of about 0.001M to about 10.0M, preferably in a concentration of about 0.01M to 0.5M, more preferably in a concentration of about 0.05M to about 0.1M. The diluting buffers will also be at a pH between about 4.5 to about 8.5, preferably at a pH between 5.5 to about 8, more preferably at a pH between 6.0 to about 7.5, still more preferably at a pH between 6.5 and about 7.3. Common biological buffers include, for example, phosphates, diphosphates, citrates, acetates, and lactates. Such buffers may also contain a nutrient source, such as for example sugar, and/or an antibiotic, such as streptomycin and combinations thereof. In one embodiment, the dilution buffer used is modified Ham's F-10 or Sperm Washing Medium (MHF-10 and SWM, respectively; Irvine Scientific, Santa Ana, Calif.). Sperm cells are somewhat sensitive to significant changes in osmotic pressure and pH. To minimize impact upon sperm viability, therefore, it is generally preferred that the dilution be formed with these considerations in mind. Consistent with these considerations, the dilution buffer can be formed in a variety of manners. The stained diluted sperm sample is then sorted in accordance to known techniques and sorted sperm cells are collected.

Although staining can lead to dilution of a sperm sample, the concentration of a stained sperm sample cannot fall—or has not been taught or suggested to fall—below about 1×10⁶ sperm cells/mL. The conventional wisdom suggests that further dilution of a sperm sample to below about 1×10⁶ sperm cells/mL leads to lower viability of the sperm cells, dye loss from the cells or difficulty in sorting process. Applicants, however, have discovered that by further dilution of a stained low volume or low concentration sperm sample, the overall sorting of viable sperm cells improves. This improvement includes sorting and collection of a higher number of viable sperm cells from diluted samples than would have been collected if the samples had not been diluted.

In one embodiment, a post-staining dilution of a low volume or low concentration sperm sample, in a 1:1 ratio or less with a diluting buffer, improves the number of sperm sorted or collected by at least 5% as compared to the number of sperm sorted when the sample was undiluted. In yet another embodiment, the stained sperm cells are diluted with a diluting buffer to obtain a dilution volume of less than about 2×10⁶ sperm cells/ml, or 1×10⁶ sperm cells/ml, or 9×10⁵ sperm cells/ml, or 7×10⁵ sperm cells/ml, or 5×10⁵ sperm cells/ml, or 3×10⁵ sperm cells/ml, or 1×10⁵ sperm cells/ml in concentration.

In one embodiment, the stained sperm sample is diluted with modified Ham's F-10 (MHF-10) or Sperm Washing Medium (SWM) (both available from Irvine Scientific, Santa Ana, Calif.). The amount of the diluting buffer employed generally depends upon several considerations, e.g., the particular buffer and the desired stained sperm concentration (# sperm cells/ml) in the dilution volume. Therefore, a sufficient amount of buffer will be used such that the desired concentration of stained sperm cells/ml is achieved. In one embodiment, buffer is added to achieve a stained sperm suspension that contains less than the concentration of sperm in the starting sperm sample. In another embodiment, buffer is added to achieve a stained sperm suspension that contains from about 1×10⁵ sperm cells/ml to about 1×10⁶ cells/ml. In another embodiment, buffer is added to achieve a stained sperm suspension that contains less than about 1×10⁵ sperm cells/ml. In yet another embodiment, buffer is added to achieve a stained sperm suspension that contains from about 5×10⁴ sperm cells/ml to about 1×10⁵. In still another embodiment, buffer is added to achieve a stained sperm suspension that contains from about 1×10⁵ sperm cells/ml to about 5×10⁵ sperm cells/ml. In another embodiment, buffer is added to achieve a stained sperm suspension that sperm cells contain from about 5×10⁵ sperm cells/ml to 9×10⁵ sperm cells/ml. In still another embodiment, buffer is added to achieve a stained sperm suspension that contains from about 9×10⁵ sperm cells/ml to about 1.4×10⁶ sperm cells/ml. In yet another embodiment, buffer is added to achieve a stained sperm suspension that contains from about 1.4×10⁶ sperm cells/ml to about 2×10⁶ sperm cells/ml.

The pH of the diluted stained sperm cell suspension is preferably maintained in the range of about 6.0 to about 8.0. More preferably, the pH of the diluted stained sperm cell suspension is maintained in the range of about 7.1 to about 7.6. Still more preferably, the pH of the diluted stained sperm cell suspension is maintained at about 7.3 to 7.4, particularly at about 7.35.

In addition to the diluting buffers, other additives may be included added to the sperm cells of the present invention to enhance the viability or motility of the sperm. Such additives include energy sources, antibiotics, protein supplements, compositions which regulate oxidation/reduction reactions intracellularly or extracellularly, motility inhibitors, motility stimulators, and seminal plasma.

Once the sperm cells are diluted according with the method of the present invention, they can be sorted and collected according to any known means that allows for separation based upon fluorescence. Commonly used and well known methods include flow cytometry systems, as exemplified by and described in U.S. Pat. Nos. 5,135,759, 5,985,216, 6,071,689, 6,149,867, and 6,263,745, as well as WO 99/33956 and WO 01/37655. Generally, the sperm sorting process is carried out by flow cytomerty and is based on differentiation of a preselected sperm cell or stain characteristic such as sex chromosome content of the sperm cell or differential fluorescence energy emission. Generally, Y-bearing sperm due to having less DNA content than X-bearing sperm are stained less and therefore emit a less intense fluorescent signal during the sorting process. This differential fluorescence intensity is utilized for sorting of sperm.

The following examples are intended to further illustrate certain preferred embodiments of the invention and are not limiting in nature. Those skilled in the art will recognize, or be able to ascertain, using no more than routine experimentation, numerous equivalents to the specific substances and procedures described herein.

### EXAMPLES

### Example 1

### Evaluation of the Effects of Dilution on Sperm Sorting

Human sperm sample dilution and sorting assay.

Materials—Human sperm samples obtained from 5 volunteers (Volunteers 1-5); modified Ham's F-10 (IMF-10), 5 mL pipets; 1 mL and 15 mL centrifuge tubes; centrifuge; Hoechst 33342 dye; diluting buffer MHF-10; and flow cytometer/cells sorter.

Methodology—Each sperm sample was prepared as follows. The semen sample was assigned a sort identification number. The semen was allowed to liquefy at 35° C. for 30 min. using 5 mL pipet, the semen was transferred from the collection vessel to a 15 mL tube then evaluated for volume, concentration, percentage motile, progression (grade 0-4), and viability (eosin dye exclusion) before processing. Semen was processed to recover motile sperm and to remove undesirable seminal components by 20 minute centrifugation at 1140 RPM (300×g) through discontinuous density gradients (ISolate, 50%, 90%, Irvine Scientific, Santa Ana, Calif., USA). After centrifugation, the supernatant was removed and recovered sperm were diluted with 6 ml buffer medium (MHF-10; Irvine Scientific, Santa Ana, Calif., USA) and centrifuged at 300×g for 10 minutes. The supernatant was removed and the sperm pellet was resuspended in buffer to achieve a sperm concentration of 40 million to 60 million sperm cells/mL. Sperm concentration, motility, progression and viability were re-evaluated after preparation. Aliquots of 10 million sperm were stained by combining the appropriate volume of sperm suspension with MHF-10 and 10 μL Hoechst 33342 (Calbiochem-Behring Corporation, La Jolla, Calif., USA) stock solution to achieve a 9 μM (final concentration) H33348 solution. After adding stain, the sperm suspension was incubated for 1 h at 37° C. For example, if the sperm concentration after preparation was 50 million per mL, then 200 μL of the sperm suspension was combined with 790 μL MHF-10. Then, 10 μL H33342 working solution was added and the sperm-stain mixture was incubated for 1 hour at 37°. Depending on the sperm concentration in the prepared sperm sample, staining conditions can be varied by adjusting the volume of the sperm suspension, the volume of the buffer used to dilute the sperm suspension, the type of buffer used, and the volume of the stain solution added. Aliquots of 200 μL or 300 μL of stained sperm cells were used to determine the effects of dilution on sperm sorting and recovery.

Regular Sort vs. Low Volume Dilution Technique—The content of each stained sperm sample (1 mL containing 10×10⁶ stained sperm) was divided into four tubes and sorted as follows: a 200 μL was aliquoted (containing 2×10⁶ sperm cells) and was sorted as control No. 1; another 200 μL (containing 2×10⁶ sperm) was aliquoted, diluted in 1:1 ratio with MHF-10 buffer, and then sorted as test No. 1; a 300 μL volume (containing 3×10⁶ sperm) was aliquoted and sorted as control No. 2; and another 300 μL volume (containing 3×10⁶ sperm) was aliquoted, diluted in 1:1 ratio with MHF-10 buffer, and then sorted as test No. 2. The data are shown in Table 1 below.

TABLE 1
Regular Sort vs. Low Volume Dilution Technique.

| | Regular | | Regular | |
|---|---|---|---|---|
| | Sort | Low volume | Sort | Low volume |
| Volunteer No. 1 | | dilution | | dilution |
| Date: Oct. 23, | Control | technique | Control | technique |
| 2008 | No. 1 | (Test No. 1) | No. 2 | (Test No. 2) |
| Volume | 200 uL | 400 uL | | 600 uL |
| | (2million) | (2million) | | (3million) |
| Motility | 80% | 80% | | 80% |
| Progression | 2.5 | 2.5 | | 2.5 |
| Purity | 73.50% | 69.50% | | 68% |
| # of cells sorted | 34,000 | 45,000 | | 67,000 |
| Volunteer No. 2 | | | | |
| Date: Oct. 27, | | dilution | | dilution |
| 2008 | | technique | | technique |
| Volume | 200 uL | 400 uL | 300 uL | 600 uL |
| | (2million) | (2million) | (3million) | (3million) |
| Motility | 80% | 80% | 80% | 80% |
| Progression | 3 | 3 | 3 | 3 |
| Purity | 81% | 84% | 71% | 85.50% |
| # of cells sorted | 15,000 | 20,000 | 30,000 | 40,000 |
| Volunteer No. 3 | | | | |
| Date: Oct. 29, | | dilution | | dilution |
| 2008 | | technique | | technique |
| Volume | 200 uL | 400 uL | 300 uL | 600 uL |
| | (2million) | (2million) | (3million) | (3million) |
| Motility | 80% | 80% | 80% | 80% |
| Progression | 2.5 | 2.5 | 2.5 | 2.5 |
| Purity | 61.50% | 66.50% | 71% | 74% |
| # of cells sorted | 35,000 | 45,000 | 50,000 | 65,000 |
| Volunteer No. 4 | | | | |
| Date: Oct. 30, | | dilution | | dilution |
| 2008 | | technique | | technique |
| Volume | 200 uL | 400 uL | 300 uL | 600 uL |
| | (2million) | (2million) | (3million) | (3million) |
| Motility | 70% | 70% | 70% | 70% |
| Progression | 2 | 2 | 2 | 2 |
| Purity | 86% | 86.50% | 90% | 87% |
| # of cells sorted | 15,000 | 22,000 | 30,000 | 41,000 |
| Volunteer No. 5 | | | | |
| Date: Nov. 4, | | dilution | | dilution |
| 2008 | | technique | | technique |
| Volume | 200 uL | 400 uL | 300 uL | 600 uL |
| | (2million) | (2million) | (3million) | (3million) |
| Motility | 80% | 80% | 80% | 80% |
| Progression | 2 | 2 | 2 | 2 |
| Purity | 78% | 72% | 74% | 75.50% |
| # of cells sorted | 10,000 | 15,000 | 25,000 | 31,000 |

Analyses of the mean and standard deviations of sorted sperm cells were performed for each set of control and test observations as shown in Table 2 below.

TABLE 2
Analysis of mean and standard deviation from data in Table 1.

| | | | | | |
|---|---|---|---|---|---|
| | Volume | Motility | | Purity | Cells |
| OBSERVATION | uL | % | Progression | % | sorted |

REGULAR SORT 2 × 10\hat{ }6 in 200 uL
Control set No. 1

| | | | | | |
|---|---|---|---|---|---|
| 1 | 200 | 80.0 | 2.5 | 73.5 | 34,000 |
| 2 | 200 | 80.0 | 3.0 | 81.0 | 15,000 |
| 3 | 200 | 80.0 | 2.5 | 61.5 | 35,000 |
| 4 | 200 | 70.0 | 2.0 | 86.0 | 15,000 |
| 5 | 200 | 80.0 | 2.0 | 78.0 | 10,000 |
| MEAN | 200 | 78 | 2.4 | 76 | 21,800 |
| SD | 0.0 | 4.5 | 0.4 | 9.3 | 11,777 |

DILUTION SORT 2 × 10\hat{ }6 IN 400 uL
Test set No. 1

| | | | | | |
|---|---|---|---|---|---|
| 1 | 400 | 80.0 | 2.5 | 69.5 | 45,000 |
| 2 | 400 | 80.0 | 3.0 | 84.0 | 20,000 |
| 3 | 400 | 80.0 | 2.5 | 66.5 | 45,000 |
| 4 | 400 | 70.0 | 2.0 | 86.5 | 22,000 |
| 5 | 400 | 80.0 | 2.0 | 72.0 | 15,000 |
| MEAN | 400 | 78 | 2.4 | 75.7 | 29,400 |
| SD | 0 | 4.5 | 0.4 | 9.0 | 14,467 |

REGULAR SORT 3 × 10\hat{ }6 in 300 uL
Control set No. 2

| | | | | | |
|---|---|---|---|---|---|
| 1 | N/A | N/A | N/A | N/A | N/A |
| 2 | 300 | 80 | 3.0 | 71.0 | 30,000 |
| 3 | 300 | 80 | 2.5 | 71.0 | 50,000 |
| 4 | 300 | 70 | 2.0 | 90.0 | 30,000 |
| 5 | 300 | 80 | 2.0 | 74.0 | 25,000 |
| MEAN | 300 | 77.5 | 2.4 | 76.5 | 33,750 |
| SD | 0.0 | 5.0 | 0.5 | 9.1 | 11,087 |

DILUTION SORT 3 × 10\hat{ }6 IN 600 uL
Test set No. 2

| | | | | | |
|---|---|---|---|---|---|
| 1 | 600 | 80.0 | 2.5 | 68.0 | 67,000 |
| 2 | 600 | 80.0 | 3.0 | 85.5 | 40,000 |
| 3 | 600 | 80.0 | 2.5 | 74.0 | 65,000 |
| 4 | 600 | 70.0 | 2.0 | 87.0 | 41,000 |
| 5 | 600 | 80.0 | 2.0 | 75.5 | 31,000 |
| MEAN | 600 | 78 | 2.4 | 78 | 48,800 |
| SD | 0.0 | 4.5 | 0.4 | 8.1 | 16,193 |

Results—The above data showed that dilution increases the number of viable sperm cells sorted by at least 134.8%. See Table 3 below.

TABLE 3
The effect of dilution on sperm sorting.

| | Volume | Motility | | | Cells | % |
|---|---|---|---|---|---|---|
| TREATMENT | uL | % | Progression | Purity % | sorted | increase |
| REGULAR SORT 2 × 10\hat{ }6 in 200 uL | 200 | 78.0 | 2.4 | 76.0 | 21,800 | |
| (n = 5) | | | | | | |
| DILUTION SORT 2 × 10\hat{ }6 in 400 uL | 400 | 78.0 | 2.4 | 75.7 | 29,400 | 134.8% |
| (n = 5) | | | | | | |
| REGULAR SORT 3 × 10\hat{ }6 in 300 uL | 300 | 77.5 | 2.4 | 76.5 | 33,750 | |
| (n = 4) | | | | | | |
| DILUTION SORT 3 × 10\hat{ }6 in 600 uL | 600 | 78.0 | 2.4 | 78.0 | 48,800 | 144.6% |
| (n = 5) | | | | | | |

What is claimed is:

1. A method of sorting sperm cells, comprising: (a) obtaining a sperm sample comprising a plurality of sperm cells in a volume of 0.3 ml or less or wherein the plurality of sperm cells is less than about 2×10⁶ cells/ml; (b) staining the sperm cells by forming a staining mixture comprising intact viable sperm cells and a DNA selective fluorescent dye for a period of time sufficient to allow the dye to bind to DNA of the sperm cells; (c) diluting the stained sperm cells of step (b) to obtain a diluted volume of said stained sperm cells; (d) sorting the stained sperm cells in the diluted volume based upon a preselected sperm cell characteristic; and (e) collecting sorted sperm cells.

2. The method of claim 1, wherein the sperm sample is freshly obtained mammalian semen orcryopreserved and subsequently thawed prior to staining.

3. The method of claim 2, wherein the sperm are obtained from a mammal selected from the group consisting of a human, bovine, equine, ovine, caprine, porcine, canine, feline, lagomorph, cavid, and marine mammals.

4. The method of claim 1, wherein the sperm sample contains less than about 1×10⁶ sperm cells/ml.

5. The method of claim 1, wherein the stained sperm mixture contains less than about 1×10⁵ sperm cells/ml.

6. The method of claim 1, wherein the diluting step comprises diluting the stained sperm cells with a buffer.

7. The method of claim 6, wherein the buffer is MHF-10 or SWM buffer.

8. The method of claim 1, wherein the preselected sperm cell characteristic is the sex chromosome content of the sperm cells.

9. The method of claim 1, wherein the fluorescent dye is selected from the group consisting of a bisbenzimide, Hoechst 33342, Hoechst 33258 and a conjugate, an analog, or a derivative thereof.

10. The method of claim 1, wherein said step (d) is performed using a flow cytometer or cell sorter.

11. The method of claim 1 wherein residual sperm cells in the sperm sample that were not sorted after completion of steps (a) through (e) are subjected to steps (c), (d) and (e) at least once.

12. A method of sorting a stained sperm sample of less than about 0.3 mL or less than 1×10⁶ sperm cells/mL, comprising: (a) diluting the stained sperm sample in a ratio of 1:1 or less by volume with a dilution buffer to obtain a diluted volume of said stained sperm sample; (b) subjecting the diluted volume of the stained sperm sample to a flow cytometric sorting process; (c) sorting the stained sperm cells in the diluted volume based upon a preselected sperm cell or stain characteristic; and (d) collecting sorted sperm cells.

13. The method of claim 12, wherein the dilution buffer is MHF-10 or SWM buffer.

14. The method of claim 12, wherein the preselected sperm cell characteristic is the sex of the sperm cells.

15. The method of claim 12, wherein said step (c) is performed using a flow cytometer or cell sorter.

16. The method of claim 12 wherein the diluted volume of sperm sample of step (a) is frozen and subsequently thawed prior to carrying out step (b).

17. The method of claim 12 wherein residual sperm cells in the stained sperm sample that were not sorted after completion of steps (a) through (d) are subjected to steps (a) through (d) at least once.
